(19) United States
(12) Patent Application Publication
(10) Pub. No.: US 2023/0411011 A1
(43) Pub. Date: Dec. 21, 2023

(54) METHOD AND DIAGNOSTIC APPARATUS FOR DETERMINING HYPERGLYCEMIA USING MACHINE LEARNING MODEL

(71) Applicant: HEM PHARMA INC., Pohang-si (KR)

(72) Inventors: Yo Sep JI, Suwon-si (KR); So Young PARK, Suwon-si (KR)

(21) Appl. No.: 18/458,297

(22) Filed: Aug. 30, 2023

Related U.S. Application Data
(63) Continuation of application No. PCT/KR2022/003896, filed on Mar. 21, 2021.

(30) Foreign Application Priority Data
Mar. 26, 2021 (KR) 10-2021-0039434

(51) Int. Cl. G16H 50/20; G06N 20/20

(52) U.S. Cl. CPC G16H 50/20; G06N 20/20

(57) ABSTRACT

A method for determining whether hyperglycemia is present by using a machine learning model may include a process of analyzing a mixture of a gut-derived substance collected from a subject and a gut environment-like composition, a process of extracting multiple microbial data based on an analysis result of the mixture, a process of selecting microbe-related features to be used in the machine learning model from the multiple microbial data based on a predetermined feature selection algorithm, a process of training the machine learning model with the microbe-related features, and a process of inputting, to the trained machine learning model, the microbial data collected from the subject to be tested and determining whether hyperglycemia is present. The microbe-related features may include the amount of one or more microbes selected from families included in orders, Oscillospirales, Lachnospirales, Lactobacillales, and Peptostreptococcales-Tissierellales.

### TECHNICAL FIELD

The present disclosure relates to a method and diagnostic apparatus for determining hyperglycemia using machine learning model.

### BACKGROUND

Hyperglycemia refers to a condition in which a blood sugar level is 180 mg/dL or more on average, and is accompanied by symptoms such as fatigue, frequent urination, feeling of hunger, dry skin and mouth, and blurred vision.

The causes of hyperglycemia include eating too much food, a diet high in carbohydrates, decreased activity, and severe stress. If hyperglycemia persists, it can develop into diabetes, and when diabetes is not well controlled, acute complications such as diabetic ketoacidosis and hyperosmolar hyperglycemic coma/syndrome can arise.

As a result of analyzing data from the National Health Insurance Service from 2004 to 2013, the number of hospitalized patients with hyperglycemic crises increased by 3,000 between 2004 and 2013. Also, the incidence and mortality according to age tended to increase with increasing age.

Currently, Korea is facing an aging society. The prevalence of diabetes among those over age 65 is continuously increasing, and the proportion of high-risk groups who are about to develop diabetes also accounts for a significant portion of patients in the elderly group.

Meanwhile, the term “genome” refers to genes present in chromosomes, the term “microbiota” refers to the collection of microbes populating an environment, and the term “microbiome” refers to the collection of all the genomes of these microbes in the environment. Here, the microbiome may refer to the combination of genome and microbiota.

Recently, there has been an attempt to diagnose hyperglycemia by identifying a microbe that can act as a causative agent of hyperglycemia through metagenome analysis of microbiota.

In this regard, Korean Patent No. 10-2057047, one of the prior art references, relates to a disease prediction apparatus and a disease prediction method using the same, and discloses a method for predicting a disease of a predetermined person by comparing a learning vector with a predetermined person vector extracted from a biosignal of the predetermined person.

However, according to the prior art reference, bacterial metagenome analysis is performed without any special process, such as sample culturing, and it is difficult to accurately derive a causative agent of hyperglycemia due to a large bias among samples of each subject.

Further, when a machine learning model is trained using unprocessed samples of each subject as training data, the training data contain a large amount of noise, which causes a significant degradation in performance of the machine learning model.

### DISCLOSURE OF THE INVENTION

### Problems to be Solved by the Invention

The present disclosure is conceived to solve the above-described problems and improve the performance of a machine learning model for diagnosing hyperglycemia by selecting microbe-related features from multiple microbial databased on an analysis result of a mixture of a sample and a gut environment-like composition.

The problems to be solved by the present disclosure are not limited to the above-described problems. There may be other problems to be solved by the present disclosure.

### Means for Solving the Problems

To solve the problems, one example of the present disclosure provides a method for diagnosing the presence or absence of hyperglycemia by using a machine learning model, comprising: a process of analyzing a mixture of a gut-derived substance collected from a subject and a gut environment-like composition, a process of extracting multiple microbial data based on an analysis result of the mixture, a process of selecting microbe-related features to be used in the machine learning model from the multiple microbial data, a process of training the machine learning model with the microbe-related features to predict whether hyperglycemia is present for each of the microbial data and a process of inputting, to the trained machine learning model, the microbial data extracted based on the analysis result of the mixture of the gut-derived substance collected from the subject to be tested and the gut environment-like composition and determining whether hyperglycemia is present based on an output value of the machine learning model, wherein the microbe-related features include the amount of one or more microbes selected from families included in orders, Oscillospirales, Lachnospirales, Lactobacillales, and Peptostreptococcales-Tissierellales.

Also, another example of the present disclosure provides an apparatus for diagnosing hyperglycemia by using a machine learning model, comprising: a microbial data extraction unit that extracts multiple microbial data based on an analysis result of a mixture of a gut-derived substance collected from a subject and a gut environment-like composition, a feature selection unit that selects microbe-related features to be used in the machine learning model from the multiple microbial data training unit that trains the machine learning model with the microbe-related features to predict whether hyperglycemia is present for each of the microbial data and a diagnosis unit that inputs, to the trained machine learning model, the microbial data extracted based on the analysis result of the mixture of the gut-derived substance collected from the subject to be tested and the gut environment-like composition and diagnoses hyperglycemia based on whether hyperglycemia is present, which is an output value of the machine learning model, wherein the microbe-related features include the amount of one or more microbes selected from families included in orders, Oscillospirales, Lachnospirales, Lactobacillales, and Peptostreptococcales-Tissierellales.

The above-described problem-solving means are merely illustrative and should not be interpreted as limiting the present invention. In addition to the above-described exemplary embodiments, additional embodiments described in the drawings and the detailed description of the invention may exist.

### Effects of the Invention

According to any one of the above-described means for solving the problems of the present disclosure, it is possible to improve the performance of a machine learning model for diagnosing hyperglycemia by selecting microbe-related features from multiple microbial data based on an analysis result of a mixture of a gut-derived substance and a gut environment-like composition.

### BRIEF DESCRIPTION OF THE DRAWINGS

FIG. 1 is a block diagram illustrating a diagnostic apparatus according to an example of the present disclosure.

FIG. 2 is a diagram illustrating an MCMOD technique according to an example of the present disclosure.

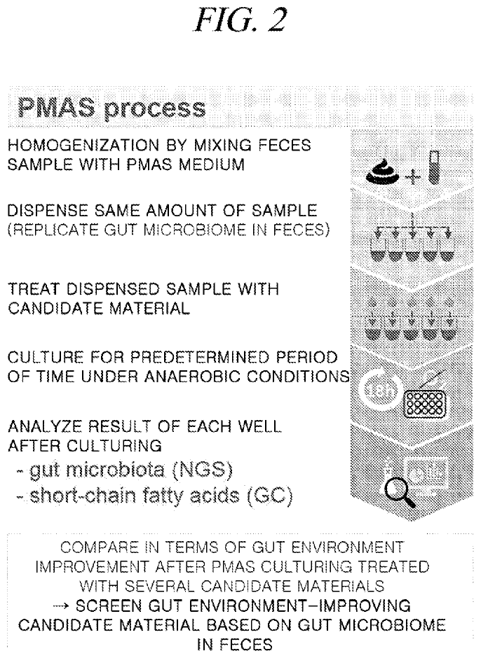

FIG. 3 is a diagram for explaining a sample analysis through the MCMOD technique according to an example of the present disclosure.

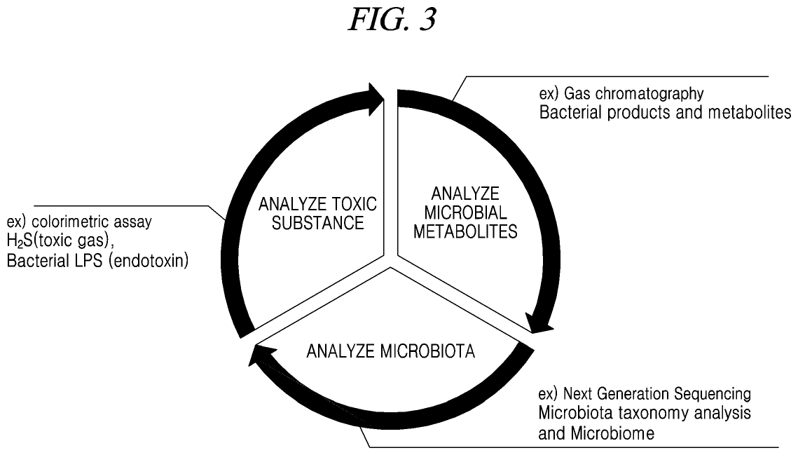

FIG. 4 is a diagram for explaining the interpretation of a sample analysis result through the MCMOD technique according to an example of the present disclosure.

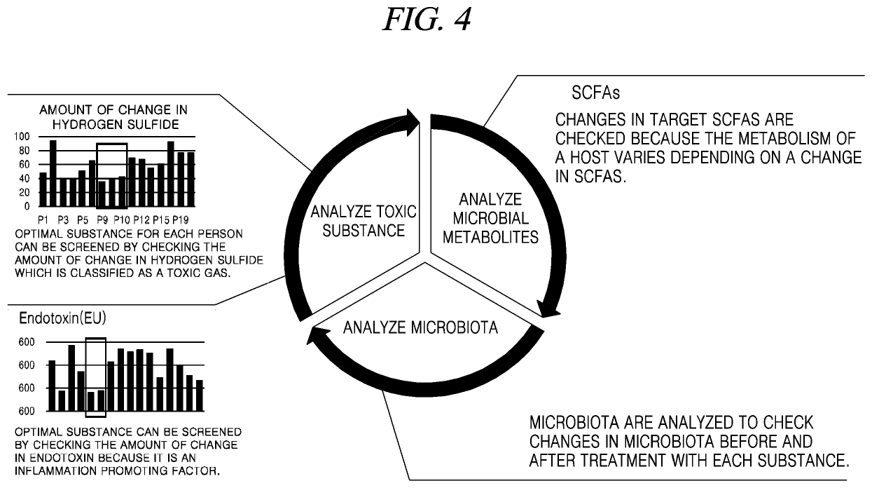

FIG. 5A is a diagram for explaining selected microbe-related features according to an example of the present disclosure.

FIG. 5B is a diagram for explaining selected microbe-related features according to an example of the present disclosure.

FIG. 5C is a diagram for explaining selected microbe-related features according to an example of the present disclosure.

FIG. 6A is a diagram comparing analysis results of respective samples according to a method for diagnosing hyperglycemia of an example of the present disclosure and a method for Comparative Example.

FIG. 6B is a diagram comparing analysis results of respective samples according to a method for diagnosing hyperglycemia of an example of the present disclosure and a method for Comparative Example.

FIG. 6C is a diagram comparing analysis results of respective samples according to a method for diagnosing hyperglycemia of an example of the present disclosure and a method for Comparative Example.

FIG. 7A is a diagram comparing analysis results of respective samples according to the method for diagnosing hyperglycemia of an example of the present disclosure and the method for Comparative Example.

FIG. 7B is a diagram comparing analysis results of respective samples according to the method for diagnosing hyperglycemia of an example of the present disclosure and the method for Comparative Example.

FIG. 8A is a diagram comparing machine learning models in performance according to the method for diagnosing hyperglycemia of an example of the present disclosure.

FIG. 8B is a diagram comparing machine learning models in performance according to the method for diagnosing hyperglycemia of Comparative Example.

FIG. 9 is a diagram illustrating changes in performance of machine learning models depending on the number of features according to the method for diagnosing hyperglycemia of an example of the present disclosure and the method for Comparative Example.

FIG. 10A is a diagram comparing random forest models in performance according to the method for diagnosing hyperglycemia of an example of the present disclosure.

FIG. 10B is a diagram comparing random forest models in performance according to the method for diagnosing hyperglycemia of Comparative Example.

FIG. 11A is a diagram comparing XGB models in performance according to the method for diagnosing hyperglycemia of an example of the present disclosure.

FIG. 11B is a diagram comparing XGB models in performance according to the method for diagnosing hyperglycemia of an example of Comparative Example.

FIG. 12 is a flowchart illustrating a method for diagnosing hyperglycemia according to an example of the present disclosure.

### BEST MODE FOR CARRYING OUT THE INVENTION

A Hereafter, embodiments of the present disclosure will be described in detail with reference to the accompanying drawings so that the present disclosure may be readily implemented by a person with ordinary skill in the art. However, it is to be noted that the present disclosure is not limited to the embodiments but may be embodied in various other ways. In drawings, parts irrelevant to the description are omitted for the simplicity of explanation, and like reference numerals denote like parts through the whole document.

Throughout this document, the term “connected to” may be used to designate a connection or coupling of one element to another element and includes both an element being “directly connected” another element and an element being “electronically connected” to another element via another element. Further, it is to be understood that the terms “comprises,” “includes,” “comprising,” and/or “including” means that one or more other components, steps, operations, and/or elements are not excluded from the described and recited systems, devices, apparatuses, and methods unless context dictates otherwise; and is not intended to preclude the possibility that one or more other components, steps, operations, parts, or combinations thereof may exist or may be added.

Throughout the whole document, the term “unit” includes a unit implemented by hardware or software and a unit implemented by both of them. One unit may be implemented by two or more pieces of hardware, and two or more units may be implemented by one piece of hardware.

In the present specification, some of operations or functions described as being performed by a device may be performed by a server connected to the device. Likewise, some of operations or functions described as being performed by a server may be performed by a device connected to the server.

Hereinafter, embodiments of the present disclosure will be described in detail with reference to the accompanying drawings.

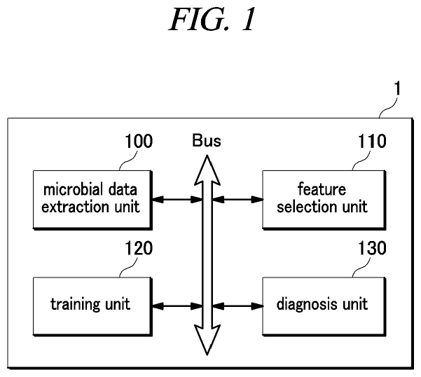

FIG. 1 is a block diagram illustrating a diagnostic apparatus according to an example of the present disclosure. Referring to FIG. 1, a diagnostic apparatus 1 may include a microbial data extraction unit 100, a feature selection unit 110, a training unit 120, and a diagnosis unit 130.

Examples of the diagnostic apparatus 1 may include a personal computer such as a desktop computer or a laptop computer, as well as a mobile device capable of wired/wireless communication. The mobile device is a wireless communication device that ensures portability and mobility and may include a smartphone, a tablet PC, a wearable device and various kinds of devices equipped with a communication module such as Bluetooth (BLE, Bluetooth Low Energy), NFC, RFID, ultrasonic waves, infrared rays, Wi-Fi, Li-Fi, and the like. However, the diagnostic apparatus 1 is not limited to the shape illustrated in FIG. 1 or the above examples.

The diagnostic apparatus 1 may detect a biomarker for diagnosing the hyperglycemia caused by abnormalities in the gut environment in a sample collected from a subject.

For example, the diagnostic apparatus 1 may diagnose the hyperglycemia based on a sample preparation process, a sample pretreatment process, a sample analysis process, a data analysis process, and derived data.

In an embodiment, the biomarker may be a substance detected in the gut, and specifically, it may include microbiota, endotoxins, hydrogen sulfide, gut microbial metabolites, short-chain fatty acids and the like, but is not limited thereto.

The microbial data extraction unit 100 may extract multiple microbial data based on an analysis result of a mixture of a sample collected from a subject and a gut environment-like composition. Herein, the multiple microbial data may be classified into a training set to be used for training and a test set, and a classification ratio may vary, such as 9:1, 7:3, 5:5 and the like, and may be preferably 7:3.

According to the present disclosure, pretreatment for analyzing a mixture of a sample and a gut environment-like composition is performed. In the present disclosure, the pretreatment may be referred to as MCMOD (Meta-culture Multi-Omics Diagnose).

For example, an in-vitro analysis of fecal microbiome and metabolites is performed to feces samples obtained from humans and various animals that can most easily represent the gut microbial environment in vivo.

Herein, the term “subject” refers to any living organism which may have a gut disorder, may have a disease caused by a gut disorder or develop it or may be in need of an improvement of gut environment. Specific examples thereof may include, but not limited to, mammals such as mice, monkeys, cattle, pigs, minipigs, domestic animals and humans, birds, cultured fish, and the like.

The term “sample” refers to a material derived from the subject and specifically may be cells, urine, feces, or the like, but may not be limited thereto as long as a material, such as microbiota, gut microbial metabolites, endotoxins and short-chain fatty acids, present in the gut can be detected therefrom.

The term “gut environment-like composition” may refer to a composition prepared for mimicking identically/similarly mimicking the gut environment of the subject in vitro. For example, the gut environment-like composition may be a culture medium composition, but is not limited thereto.

The gut environment-like composition may include L-cysteine hydrochloride and mucin.

Herein, the term “L-cysteine hydrochloride” is one of amino acid supplements and plays an important role in metabolism as a component of glutathione in vivo and is also used to inhibit browning of fruit juices and oxidation of vitamin C.

L-cysteine hydrochloride may be contained at a concentration of, for example, from (w/v) to 5% (w/v), specifically from 0.01% (w/v) to 0.1% (w/v).

L-cysteine hydrochloride is one of various formulations or forms of L-cysteine, and the composition may include L-cysteine including other types of salts as well as L-cysteine.

The term “mucin” is a mucosubstance secreted by the mucous membrane and includes submandibular gland mucin and others such as gastric mucosal mucin and small intestine mucin. Mucin is one of glycoproteins and known as one of energy sources such as carbon sources and nitrogen sources that gut microbiota can actually use.

Mucin may be contained at a concentration of, for example, 0.01% (w/v) to 5% (w/v), specifically, from 0.1% (w/v) to 1% (w/v), but is not limited thereto.

In an embodiment, the gut environment-like composition may not include any nutrient other than mucin and specifically may not include a nitrogen source and/or carbon source such as protein and carbohydrate.

The protein that serves as a carbon source and nitrogen source may include one or more of tryptone, peptone and yeast extract, but may not be limited thereto. Specifically, the protein may be tryptone.

The carbohydrate that serves as a carbon source may include one or more of monosaccharides such as glucose, fructose and galactose and disaccharides such as maltose and lactose, but may not be limited thereto. Specifically, the carbohydrate may be glucose.

In an embodiment, the gut environment-like composition may not include glucose and tryptone, but is not limited thereto.

The gut environment-like composition may further include one or more selected from the group consisting of sodium chloride (NaCl), sodium carbonate (NaHCO₃), potassium chloride (KCl) and hemin. Specifically, sodium chloride may be contained at a concentration of, for example, from 10 mM to 100 mM, sodium carbonate may be contained at a concentration of, for example, from 10 mM to 100 mM, potassium chloride may be contained at a concentration of, for example, from 1 mM to 30 mM, and hemin may be contained at a concentration of, for example, from 1×10⁻⁶ g/L to 1×10⁻⁴ g/L, but is not limited thereto.

In the pretreatment, the mixture may be cultured for 18 to 24 hours under anaerobic conditions.

For example, in an anaerobic chamber, the same amount of a homogenized feces-medium mixture is dispensed to each of culture plates such as 96-well plates. Herein, the culture may be performed for 12 hours to 48 hours, specifically, for 18 hours to 24 hours, but is not limited thereto.

Then, the plates are cultured under anaerobic conditions with temperature, humidity and motion similar to those of the gut environment to ferment and culture the respective test groups.

After the culturing of the mixture, a culture in which the mixture has been cultured is analyzed. The analysis of the culture may be to extract microbial data including at least one of the content, concentration and kind of one or more of endotoxins, hydrogen sulfides, short-chain fatty acids (SCFAs) and microbiota-derived metabolites contained in the culture, and a change in kind, concentration, content or diversity of bacteria included in the microbiota, but is not limited thereto.

Herein, the term “endotoxin” is a toxic substance that can be found inside a bacterial cell and acts as an antigen composed of a complex of proteins, polysaccharides, and lipids. In an embodiment, the endotoxin may include lipopolysaccharides (LPS), but may not limited thereto, and the LPS may be specifically gram negative and pro-inflammatory.

The term “short-chain fatty acid (SCFA)” refers to a short-length fatty acid with six or fewer carbon atoms and is a representative metabolite produced from gut microbiota. The SCFA has useful functions in the body, such as an increase in immunity, stabilization of gut lymphocytes, a decrease in insulin signaling, and stimulation of sympathetic nerves.

In an embodiment, the short-chain fatty acids may include one or more selected from the group consisting of formate, acetate, propionate, butyrate, isobutyrate, valerate and iso-valerate, but may not be limited thereto.

The culture may be analyzed by various analysis methods, such as genetic analysis methods including absorbance analysis, chromatography analysis and next generation sequencing, and metagenomic analysis methods, that can be used by a person with ordinary skill in the art.

When the culture is analyzed, the culture may be centrifuged to separate a supernatant and a precipitate and then, the supernatant and the precipitate (pallet) may be analyzed. For example, metabolites, short-chain fatty acids, toxic substances, etc. from the supernatant and microbiota from the pallet may be analyzed.

For example, after the culturing is completed, toxic substances, such as hydrogen sulfide and bacterial LPS (endotoxin), microbial metabolites, such as short-chain fatty acids, from the supernatant obtained by centrifugation of the cultured test groups are analyzed through absorbance analysis and chromatography analysis, and a culture-independent analysis method is performed to the microbiota from the centrifuged pellet. For example, the amount of change in hydrogen sulfide produced by the culturing may be measured through a methylene blue method using N,N-dimethyl-p-phenylene-diamine and iron chloride (FeCl₃) and the level of endotoxins that is one of inflammation promoting factors may be measured using an endotoxin assay kit. Also, microbial metabolites such as short-chain fatty acids including acetate, propionate and butyrate can be analyzed through gas chromatography.

Microbiota can be analyzed by genome-based analysis through metagenomic analysis such as real-time PCR in which all genomes are extracted from a sample and a bacteria-specific primer suggested in the GULDA method or next generation sequencing.

According to the present disclosure, the culture is analyzed in a state where the gut environment is implemented in vitro by using the gut environment-like composition, and, thus, it is possible to reduce a bias between training data by optimizing the training data before machine learning.

Accordingly, it is possible to facilitate selection of microbe-related features to be described later and also improve the performance of a machine learning model by training the machine learning model based on the microbe-related features. Therefore, it is possible to increase the accuracy in diagnosing the hyperglycemia through the trained machine learning model.

The feature selection unit 110 may perform selection (i.e., feature selection) of microbe-related features from multiple microbial data as features to be used for the machine learning model based on a predetermined feature selection algorithm. The number of the microbe-related features may be 6 to 10. For example, the number of the microbe-related features may be 7.

Features (variables or attributes) are used in creating a machine learning model. If a large number of features or inappropriate features are used, the machine learning model may overfit data or the prediction accuracy may decrease.

Accordingly, in order for the machine learning model to have a high prediction accuracy, it is necessary to use an appropriate combination of features. That is, it is possible to reduce the complexity of the machine learning model while using as few features as possible by selecting features most closely related to a response feature to be predicted.

The feature selection algorithm may include at least one of, for example, a Boruta algorithm and a recursive feature elimination (RFE) algorithm.

The microbe-related features selected from a predetermined feature selection algorithm may include the amount of one or more microbes selected from families included in orders, Oscillospirales, Lachnospirales, Lactobacillales, and Peptostreptococcales—Tissierellales.

In an embodiment, the microbe-related features selected from the predetermined feature selection algorithm may further include the amount of one or more microbes selected from genera included in families, for example, Ruminococcaceae, Lachnospiraceae, Leuconostocaceae, and Peptostreptococcaceae.

In an embodiment, the microbe-related features selected from the predetermined feature selection algorithm may further include the amount of one or more microbes selected from species included in genera, for example, Subdoligranulum, Ruminococcus, Weissella, and Intestinibacter.

The training unit 120 may train the machine learning model with the microbe-related features.

For example, the training unit 120 may train machine learning model to predict whether hyperglycemia is present for each of microbial data by performing supervised learning based on labeling of whether hyperglycemia is present for each of the microbial data (learning data) and the amount of microbes related to the selected feature.

The machine learning model may include at least one of, for example, a logistic regression model, a generalized linear (GLM) model, a random forest model, a gradient boosting model, and an extreme gradient boosting (XGB) model.

The diagnosis unit 130 may diagnose hyperglycemia by inputting, to the trained machine learning model, the microbial data extracted based on the analysis result of the mixture of the gut-derived substance collected from the subject to be tested and the gut environment-like composition.

For example, the diagnosis unit 130 may diagnose hyperglycemia based on whether hyperglycemia is present, which is an output value of the machine learning model. That is, the diagnosis unit 130 may determine whether the subject to be tested has hyperglycemia or predict the incidence of hyperglycemia of the subject to be tested based on the output value of the machine learning model.

Hereinafter, Examples of the present disclosure will be described in detail. However, the present disclosure is not limited thereto.

### EXAMPLES

### Example 1. Microbe-Related Feature Selected Based on Recursive Feature Elimination Algorithm after or without MCMOD Treatment

In order to check microbe-related features selected based on a recursive feature elimination algorithm after or without MCMOD treatment of Example 1, a test was performed as follows.

According to the present disclosure, a pre-treatment is performed to analyze a mixture of a sample and a gut environment-like composition. In the present disclosure, the above-described pre-treatment may be referred to as MCMOD. Meanwhile, in the present disclosure, Comparative Example relates to a method for determining hyperglycemia based on microbial data extracted by performing only a conventional pre-treatment without performing the above-described pre-treatment on a sample. In this regard, the conventional pretreatment for Comparative Example is referred to as SMOD.

As shown in Table 1 below, samples were microbial data from MCMOD and SMOD of a simple clinical data set (feces) based on questionnaire results received from 55 hyperglycemia patients (disease group) and 56 normal people (normal group). In particular, oversampling was performed on the data set to reduce class imbalance, and the data set was transformed into a total of 126 data sets including 63 normal data and 63 obesity data.

TABLE 1

| | | | | Number of Samples from | | | Original Data | | |
|---|---|---|---|---|---|---|---|---|---|
| Disease and | | Data Source | | Original Data | | | Train Set | | |
| Examination | | (Collection | Criteria | Disease | Normal | | Disease | Normal | |
| Item | Classification | Route) | for Disease | Group | Group | Total | Group | Group | Total |
| Hyperglycemia | Test Result | Gibbeum | More than | 55 | 56 | 111 | 41 | 43 | 84 |
| | Sheet | Hospital | 100 mg/dL of | | | | | | |
| | | | fasting blood | | | | | | |
| | | | sugar | | | | | | |

| | Original Data | | | Oversampling | | | | | |
|---|---|---|---|---|---|---|---|---|---|
| Disease and | Test Set | | | Train Set | | | Test Set | | |
| Examination | Disease | Normal | | Disease | Normal | | Disease | Normal | |
| Item | Group | Group | Total | Group | Group | Total | Group | Group | Total |

| | | | | | | | |
|---|---|---|---|---|---|---|---|
| Hyperglycemia | 14 | 13 | 27 | 63 | 63 | 126 | — |

Microbial data were classified into training data (Train set) to be used for learning and test data (Test set) at a ratio of 7:3.

Then, feature selection was performed on the training data through the Boruta algorithm and the recursive feature elimination algorithm to select microbe-related features to be used in the machine learning model. Meanwhile, as will be described below, the test data were used to assess the performance of the machine learning model.

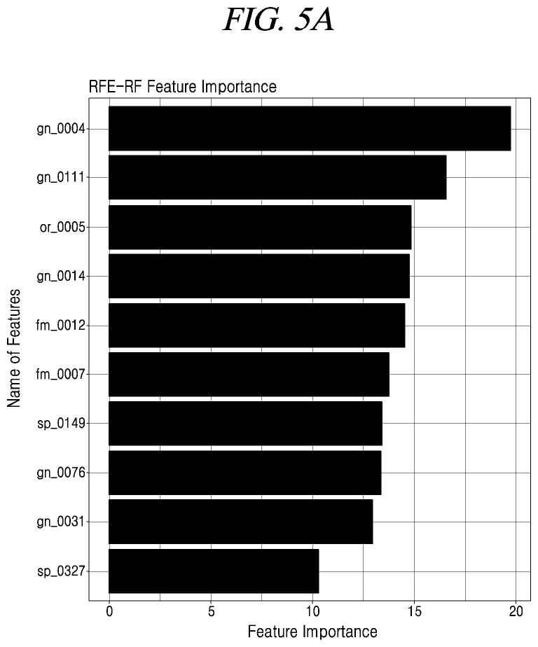

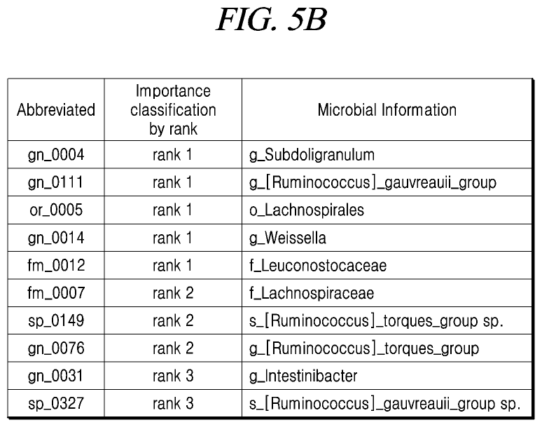

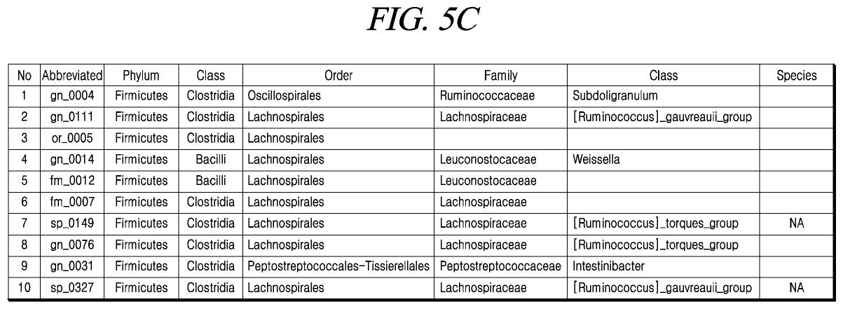

FIG. 5A, FIG. 5B and FIG. 5C are diagrams for explaining selected microbe-related features according to an example of the present disclosure.

The recursive feature elimination algorithm was used to select 10 microbe-related features in Example and 32 microbe-related features in Comparative Example as a feature group with the highest accuracy. FIG. 5A shows the importance (accuracy) of the microbe-related features selected in Example of the present disclosure, and FIG. 5B shows the microbe-related features selected in Example of the present disclosure.

Also, FIG. 5C shows taxonomic information of the microbe-related features selected in Example of the present disclosure.

In FIG. 5B and FIG. 5C, an alphabet letter in an abbreviation refers to a taxonomic rank. That is, ‘p’ stands for Phylum, ‘c’ stands for Class, ‘o’ stands for Order, ‘f’ stands for Family, ‘g’ stands for Genus, and ‘s’ stands for Species.

In FIG. 5B and FIG. 5C, the abbreviations were made arbitrarily.

For example, in the MCMOD, a microbe-related feature with high accuracy among the multiple selected microbe-related features may be a microbe belonging to the family Ruminococcaceae in the order Oscillospirales.

### Comparative Example 1. Analysis Results of Feces Samples Treated with MCMOD and Feces Samples not Treated with MCMOD

Feces were collected from one subject for 8 days, and 8 feces samples (J01, J02, J03, J04, J06, J08, J09 and J10) sorted by date were treated with MCMOD and then subjected to next-generation sequencing to analyze genes of microbes (Example). Similarly, feces samples not treated with MCMOD were subjected to next-generation sequencing to analyze genes of microbes (Comparative Example).

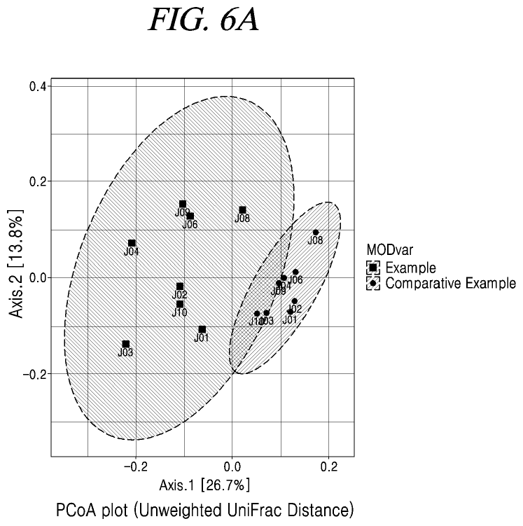

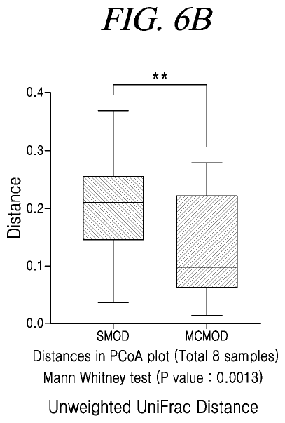

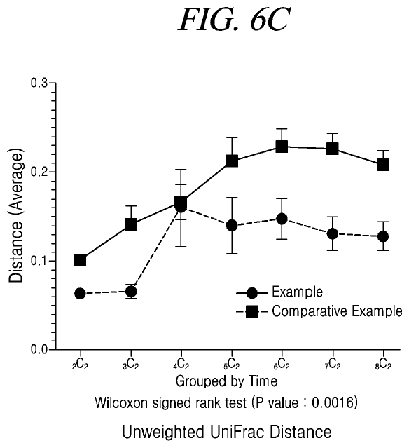

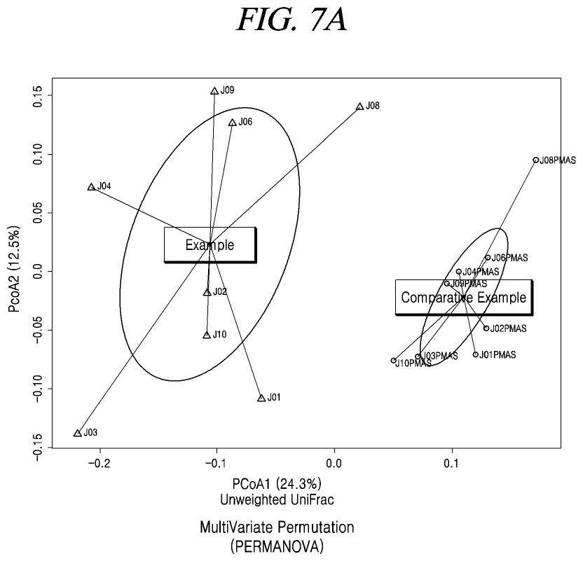

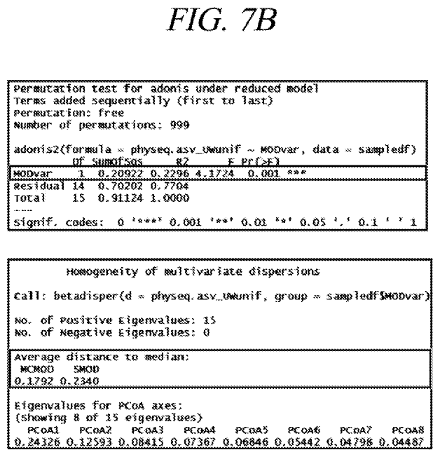

FIG. 6A, FIG. 6B, and FIG. 6C are diagrams comparing analysis results of respective samples according to a method for diagnosing hyperglycemia of an example of the present disclosure and a method for Comparative Example, and FIG. 7A and FIG. 7B are diagrams comparing analysis results of respective samples according to the method for diagnosing hyperglycemia of an example of the present disclosure and the method for Comparative Example.

FIG. 6A shows, as a PCoA plot, the beta diversity of the feces sample by using the Unweighted Unifrac Distance. As shown in the PCoA plot of FIG. 6A, it can be seen that the feces samples treated with MCMOD are relatively clustered, whereas the feces samples not treated with MCMOD are relatively scattered.

FIG. 6B shows, as a box plot, the distances among 8 points in each group (Example and Comparative Example) on the PCoA plot.

As can be seen from the box plot, the differences among the feces samples of Example are statistically significantly smaller than those of Comparative Example.

FIG. 6C shows the distances among 8 points in each group (Example and Comparative Example) on the PCoA plot.

Since there are 8 samples in each group, each group has a total of 28 types of distances between two samples. The samples with 28 types of distances were grouped in chronological order from ₂C₂ to ₈C₂.

Since a feces sample J01 was collected first and a feces sample J10 was collected last, the distance between the two samples collected first and second in the group ₂C₂ (N=1) (the distance between the samples J01 and J02) was calculated.

In the group ₃C₂ (N=3), the distances among the three samples including the next collected feces sample J03 (between J01 and J02, between J01 and J03, and between J02 and J03) were calculated to find the average and standard error of the distances.

In the group ₄C₂ (N=6), the distances among the four samples including the next collected feces sample J04 (between J01 and J02, between J01 and J03, between J01 and J04, between J02 and J03, between J02 and J04, and between J03 and J04) were calculated to find the average and standard error of the distances.

Similarly, in the group ₈C₂ (N=28), the distances among the eight samples including the last collected feces sample J10 (a total of 28 types of distances) were calculated to find the average and standard error of the distances.

As can be seen from the distance values in the PCoA plot, the differences among the feces sample groups (₂C₂ to ₈C₂) of Example are statistically significantly smaller than those of Comparative Example.

FIG. 7A and FIG. 7B show analysis results of the two groups (Example and Comparative Example) through PERMANOVA tests.

Based on the result of PERMANOVA tests as shown in FIG. 7B, a Pr(>F) value is as small as 0.001, which indicates that the two groups (Example and Comparative Example) are different in terms of population mean. This means there is a statistically significant difference between the two groups.

Also, it can be seen that the average distance to median of each feces sample in each group is smaller in Example (0.1792) than in Comparative Example (0.2340), which means that Example has less noise than Comparative Example.

As described above, the feces samples treated with MCMOD have relatively little noise due to a small bias between the feces samples and thus have low fluctuations.

That is, according to the present disclosure, the feces samples are treated with MCMOD before feature selection and machine learning training to facilitate feature selection, and, as will be described later, the machine learning model is trained to improve the performance of the machine learning model.

### Comparative Example 2. Comparison of Performance Between Machine Learning Models Trained with Training Data Obtained from MCMOD-Treated Fecal Sample and Non-Treated Fecal Sample, Respectively

The fecal sample collected in Example 1 was subjected to the MCMOD to extract microbial data (Example), and microbial data were extracted without the MCMOD (Comparative Example).

The recursive feature elimination algorithm was used to select 10 microbe-related features from the microbial data in Example and 32 microbe-related features from the microbial data in Comparative Example.

The microbe data and microbe-related features of Example and Comparative Example were used to train each of a logistic regression analysis (LRA) model, a random forest (RF) model, a GLM model, a gradient boosting model, and an XGB model and then, the performance of each machine learning model was assessed.

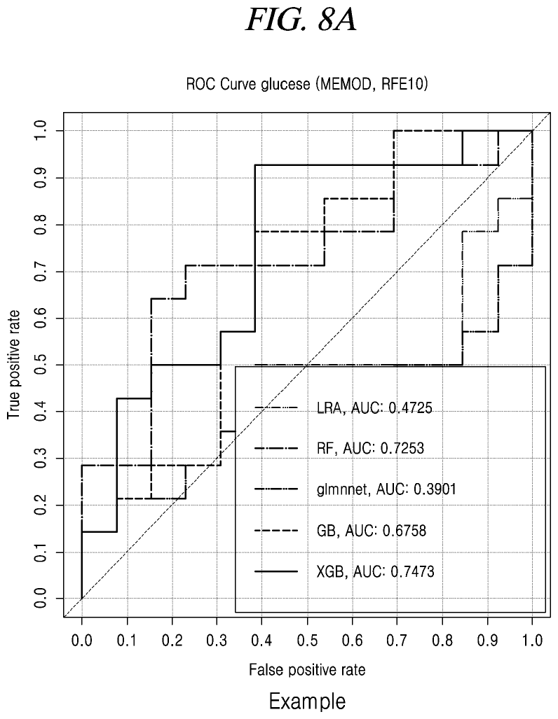

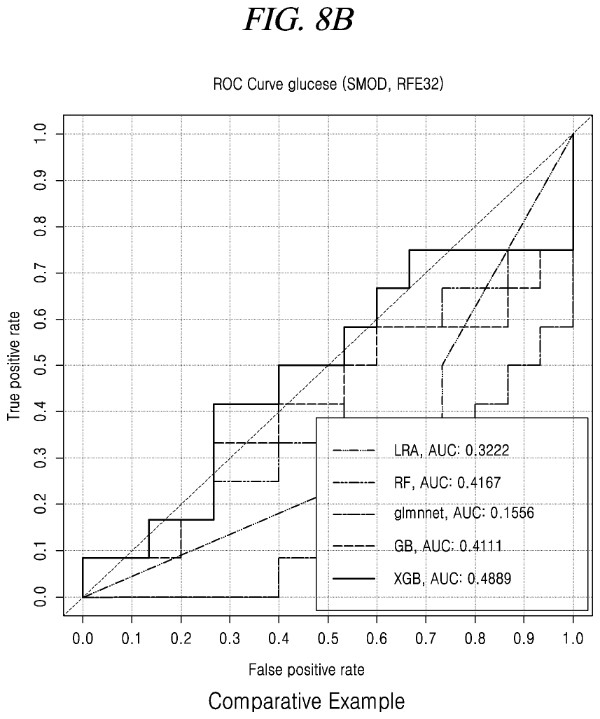

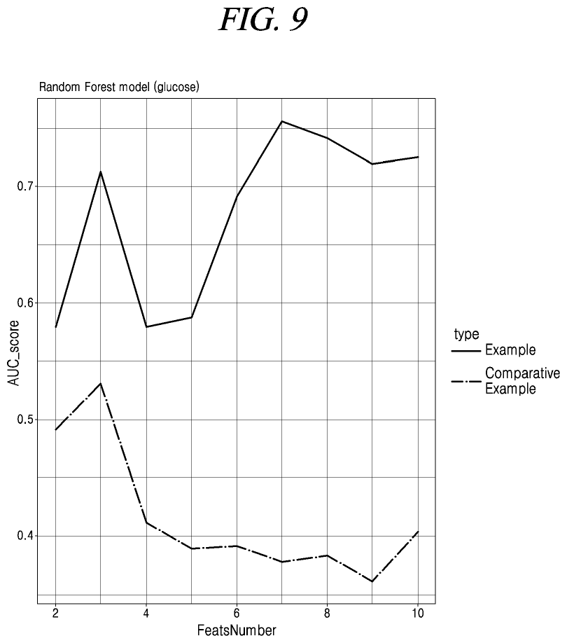

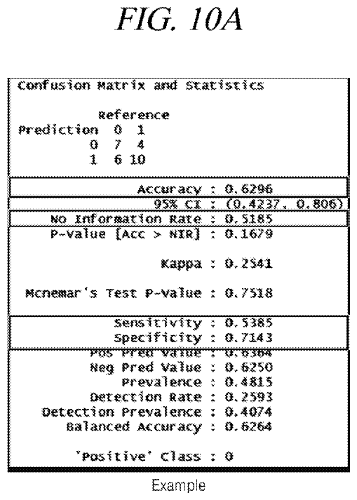

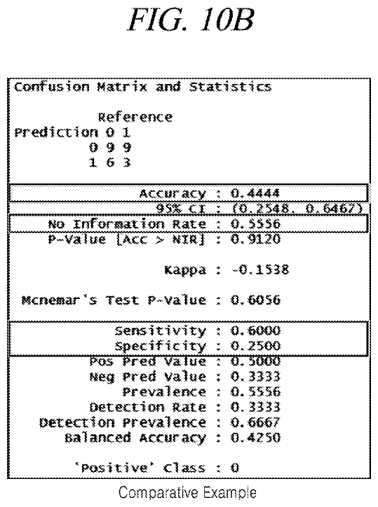

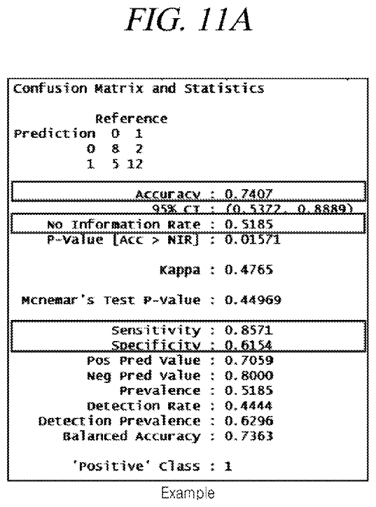

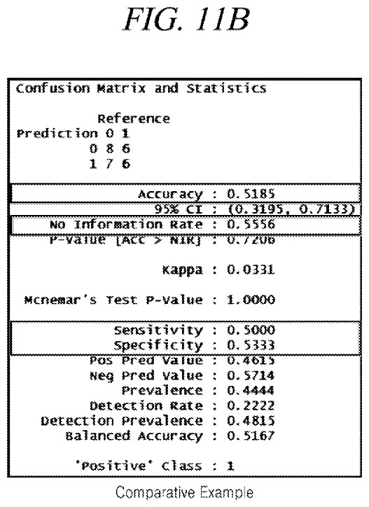

FIG. 8 and FIG. 8B are diagrams comparing the machine learning models in terms of performance according to a hyperglycemia diagnosis method for Example of the present disclosure and a method for Comparative Example, FIG. 9 is a diagram showing changes in performance of the machine learning models depending on the number of features according to the hyperglycemia diagnosis method for Example of the present disclosure and the method for Comparative Example, FIG. 10A and FIG. 10B are diagrams comparing random forest models in terms of performance according to the hyperglycemia diagnosis method for Example of the present disclosure and the method for Comparative Example, and FIG. 11A and FIG. 11B are diagrams comparing XGB models in terms of performance according to the hyperglycemia diagnosis method for Example of the present disclosure and the method for Comparative Example.

FIG. 8A and FIG. 8B show an ROC curve and AUC scores for each machine learning model. As shown in FIG. 8A and FIG. 8B, it can be seen that when machine learning models were trained with the microbial data of Example, all the machine learning models of Example had higher performance than those of Comparative Example. Here, as shown in FIG. 9, it can be seen that in Example, the performance of the machine learning model was the highest when 7 features were selected.

FIG. 10A and FIG. 10B show the accuracy, sensitivity and specificity of the random forest model trained with the microbial data of Example and the random forest model trained with the microbial data of Comparative Example, and FIG. 11A and FIG. 11B show the accuracy, sensitivity and specificity of the XGB model trained with the microbial data of Example and the XGB model trained with the microbial data of Comparative Example.

Herein, No Information Rate indicates the accuracy when a prediction is made collectively for one group (disease or normal) in the test set. For example, if a disease group is composed of 6 patients and a test group is composed of 4 people in the test set, No Information Rate is 0.6 when a predication is made for the disease group in the test set.

As shown in FIG. 10A, FIG. 10B, FIG. 11A and FIG. 11B, it can be seen that the machine learning model trained with the microbial data of Example has higher accuracy, sensitivity and specificity than the machine learning model trained with the microbial data of Comparative Example.

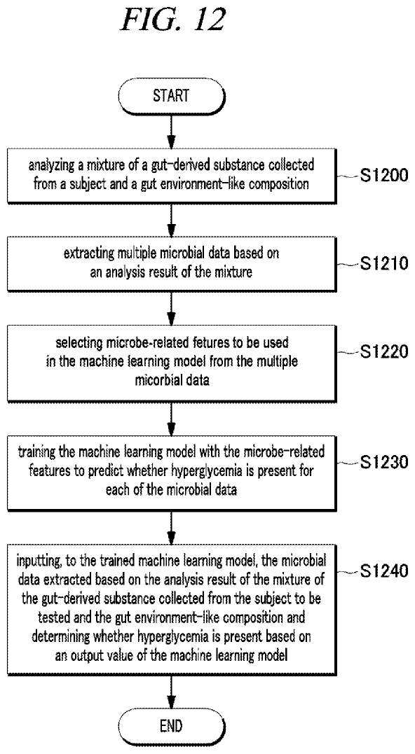

FIG. 12 is a flowchart illustrating a method for diagnosing hyperglycemia according to an example of the present disclosure. The method for diagnosing hyperglycemia according to the example illustrated in FIG. 12 includes the processes time-sequentially performed by the diagnostic apparatus illustrated in FIG. 1. Therefore, the above descriptions of the processes may also be applied to the method for diagnosing hyperglycemia according to the example illustrated in FIG. 12, even though they are omitted hereinafter.

Referring to FIG. 12, a mixture of a gut-derived substance collected from a subject and a gut environment-like composition may be analyzed in a process S1200.

In a process S1210, multiple microbial data may be extracted based on an analysis based on an analysis result of the mixture.

In a process S1220, microbe-related features to be used in the machine learning model may be selected from the multiple microbial data.

In a process S1230, the machine learning model may be trained with the microbe-related features to predict whether hyperglycemia is present for each of the microbial data.

In a process S1240, the microbial data extracted based on the analysis result of the mixture of the gut-derived substance collected from the subject to be tested and the gut environment-like composition may be input to the trained machine learning model and whether hyperglycemia is present may be determined based on an output value of the machine learning model.

Hyperglycemia can be diagnosed by inputting microbial data collected from a test subject into the trained machine learning model.

The method for diagnosing hyperglycemia illustrated in FIG. 12 can be embodied in a storage medium including instruction codes executable by a computer such as a program module executed by the computer. A computer-readable medium can be any usable medium which can be accessed by the computer and includes all volatile/non-volatile and removable/non-removable media. Further, the computer-readable medium may include all computer storage media. The computer storage media include all volatile/non-volatile and removable/non-removable media embodied by a certain method or technology for storing information such as computer-readable instruction code, a data structure, a program module or other data.

The above description of the present disclosure is provided for the purpose of illustration, and it would be understood by a person with ordinary skill in the art that various changes and modifications may be made without changing technical conception and essential features of the present disclosure. Thus, it is clear that the above-described examples are illustrative in all aspects and do not limit the present disclosure. For example, each component described to be of a single type can be implemented in a distributed manner. Likewise, components described to be distributed can be implemented in a combined manner.

The scope of the present disclosure is defined by the following claims rather than by the detailed description of the embodiment. It shall be understood that all modifications and embodiments conceived from the meaning and scope of the claims and their equivalents are included in the scope of the present disclosure.

1. A method for diagnosing the presence or absence of hyperglycemia by using a machine learning model, comprising:
a process of analyzing a mixture of a gut-derived substance collected from a subject and a gut environment-like composition;
a process of extracting multiple microbial data based on an analysis result of the mixture;
a process of selecting microbe-related features to be used in the machine learning model from the multiple microbial data based on a Boruta algorithm or a recursive feature elimination (RFE) algorithm;
a process of training the machine learning model with the microbe-related features to predict whether hyperglycemia is present for each of the microbial data; and
a process of inputting, to the trained machine learning model, the microbial data extracted based on the analysis result of the mixture of the gut-derived substance collected from the subject to be tested and the gut environment-like composition and determining whether hyperglycemia is present based on an output value of the machine learning model,
wherein the microbe-related features include the amount of one or more microbes selected from families included in orders, Oscillospirales, Lachnospirales, Lactobacillales, and Peptostreptococcales-Tissierellales.

2. The method for diagnosing the presence or absence of hyperglycemia of claim 1,
wherein the number of features to be used in the machine learning model is 6 to 10.

3. The method for diagnosing the presence or absence of hyperglycemia of claim 1, wherein the process of analyzing a mixture includes:
a process of culturing the mixture for 18 to 24 hours under anaerobic conditions; and
a process of analyzing a culture in which the mixture has been cultured.

4. The method for diagnosing the presence or absence of hyperglycemia of claim 3, wherein the process of analyzing a culture includes:
a process of centrifuging the culture to separate a supernatant and a precipitate and analyzing the supernatant and the precipitate.

5. The method for diagnosing the presence or absence of hyperglycemia of claim 3,
wherein the microbial data include at least one of the amount, concentration, and kind of one or more of endotoxins, hydrogen sulfides, short-chain fatty acids (SCFAs) and microbiota-derived metabolites contained in the culture, and a change in kind, concentration, amount or diversity of bacteria included in the microbiota.

6. The method for diagnosing the presence or absence of hyperglycemia of claim 1,
wherein the machine learning model includes at least one of a logistic regression model, a generalized linear (GLM) model, a random forest model, a gradient boosting model, and an extreme gradient boosting (XGB) model.

7. The method for diagnosing the presence or absence of hyperglycemia of claim 1,
wherein the microbe-related features include the amount of one or more microbes selected from genera included in families, Ruminococcaceae, Lachnospiraceae, Leuconostocaceae, and Peptostreptococcaceae.

8. The method for diagnosing the presence or absence of hyperglycemia of claim 1,
wherein the microbe-related features include the amount of one or more microbes selected from species included in genera, Subdoligranulum, Ruminococcus, Weissella, and Intestinibacter.

9. An apparatus for diagnosing hyperglycemia by using a machine learning model, comprising:
a microbial data extraction unit that extracts multiple microbial data based on an analysis result of a mixture of a gut-derived substance collected from a subject and a gut environment-like composition;
a feature selection unit that selects microbe-related features to be used in the machine learning model from the multiple microbial data based on a Boruta algorithm or a recursive feature elimination (RFE) algorithm;
a training unit that trains the machine learning model with the microbe-related features to predict whether hyperglycemia is present for each of the microbial data; and
a diagnosis unit that inputs, to the trained machine learning model, the microbial data extracted based on the analysis result of the mixture of the gut-derived substance collected from the subject to be tested and the gut environment-like composition and diagnoses hyperglycemia based on whether hyperglycemia is present, which is an output value of the machine learning model,
wherein the microbe-related features include the amount of one or more microbes selected from families included in orders, Oscillospirales, Lachnospirales, Lactobacillales, and Peptostreptococcales-Tissierellales.

10. The apparatus for diagnosing hyperglycemia of claim 9,
wherein the number of features to be used in the machine learning model is 6 to 10.

11. The apparatus for diagnosing hyperglycemia of claim 9,
wherein the microbial data include at least one of the amount, concentration and kind of one or more of endotoxins, hydrogen sulfides, short-chain fatty acids (SCFAs) and microbiota-derived metabolites contained in a culture in which the mixture has been cultured 18 to 24 hours under anaerobic conditions, and a change in kind, concentration, amount or diversity of bacteria included in the microbiota.

12. The apparatus for diagnosing hyperglycemia of claim 9,
wherein the machine learning model includes at least one of a logistic regression model, a generalized linear (GLM) model, a random forest model, a gradient boosting model, and an extreme gradient boosting (XGB) model.

13. The apparatus for diagnosing hyperglycemia of claim 9,
wherein the microbe-related features include the amount of one or more microbes selected from genera included in families, Ruminococcaceae, Lachnospiraceae, Leuconostocaceae, and Peptostreptococcaceae.

14. The apparatus for diagnosing hyperglycemia of claim 9,
wherein the microbe-related features include the amount of one or more microbes selected from species included in genera, Subdoligranulum, Ruminococcus, Weissella, and Intestinibacter.
